(19) United States
(12) Patent Application Publication
(10) Pub. No.: US 2013/0052673 A1
(43) Pub. Date: Feb. 28, 2013

(54) USE OF ENZYME EMULSION THICKNESS TO AFFECT CALIBRATION CODE FACTORS IN TEST STRIP MANUFACTURING

(71) Applicants: David MORRIS, Inverness (GB); Neil WHITEHEAD, Dingwall (GB); Lynsey WHYTE, Newtonmore (GB)

(72) Inventors: David MORRIS, Inverness (GB); Neil WHITEHEAD, Dingwall (GB); Lynsey WHYTE, Newtonmore (GB)

(73) Assignee: LifeScan Scotland Ltd., Inverness-shire (GB)

(21) Appl. No.: 13/218,944

(22) Filed: Aug. 26, 2011

(51) Int. Cl. C12Q 1/54

(52) U.S. Cl. 435/14

(57) ABSTRACT

The invention provides a method of controlling the slope, intercept and bias for a batch of test strips by increasing or decreasing the enzyme pad height of the strips. The invention provides an improved method for the production of single calibration code strip lots with good yields.

### FIELD OF THE INVENTION

The invention relates to test strip manufacturing for producing electrochemical test strips. In particular, the invention relates to use of the thickness of the emulsion layer used on the screen used to print test strips to control batch slope, intercept and bias to obtain a test strip batch with a desired calibration code.

### BACKGROUND OF THE INVENTION

Electrochemical test strips are designed to measure the concentration of an analyte, such as glucose, in a body fluid sample. In the case of the measurement of glucose in a blood sample, the glucose measurement is based on the selective oxidation of glucose, as for example, by the glucose oxidase enzyme. The glucose is oxidized to gluconic acid by the oxidized form of glucose oxidase and the oxidized enzyme is converted to its reduced state. Next, the reduced enzyme is re-oxidized by reaction with a mediator, such as ferricyanide. During this re-oxidation, the ferricyanide mediator is reduced to ferrocyanide.

When these reactions are conducted with a test voltage applied between two electrodes, a test current is created by the electrochemical re-oxidation of the reduced mediator at the electrode surface. Since, in an ideal environment, the amount of reduced mediator created during the chemical reaction is directly proportional to the amount of glucose in the sample positioned between the electrodes, the test current generated is proportional to the glucose content of the sample.

Test meters that use this principle enable an individual to sample and test a blood sample and determine the blood's glucose concentration at any given time. The glucose current generated is detected by the test meter and converted into a glucose concentration reading using an algorithm that relates the test current to a glucose concentration via a simple mathematical formula. In general, the test meters work in conjunction with a disposable test strip that may include a sample-receiving chamber and at least two electrodes disposed within the sample-receiving chamber in addition to the enzyme and the mediator.

Such a glucose test using a test meter and strip use batch calibration information about the test strip, such as batch slope and intercept values, determined from the manufacturing of a particular strip lot, or batch. When a user performs a glucose test using a strip from a particular strip lot, the batch slope and batch intercept information must be inputted into a test meter in the form of a calibration code by the user if the information varies batch-to-batch. If a user forgets to account for a change in calibration factors when using a different lot of test strips, there is a possibility that an inaccurate glucose measurement result may occur. Such an error can lead to insulin dose errors by the individual resulting in a hypo- or hyperglycemic episode.

To overcome this disadvantage of using test strips, test strip manufacturers have developed test strips and methods of manufacturing the strips, in which test strip lots can be prepared that do not require a user to input any calibration information before performing a test measurement because a high percentage of test strip lots can be produced that have a relatively constant batch slope and batch intercept. Thus, the test strip lots effectively have the same calibration and, when the test strips are used in a glucose test meter manufactured with the calibration information, no calibration coding is necessary or required of the user during each usage of the test strips.

### BRIEF DESCRIPTION OF THE DRAWINGS

FIG. 1 is a graph of the enzyme pad height versus screen emulsion thickness.

FIG. 2 is a graph of intercept and slope plotted against enzyme pad height

FIG. 3 is a scatterplot graph of bias versus glucose level if the test strip batches of Example 1.

### DETAILED DESCRIPTION OF THE INVENTION

It is a discovery of the invention that the slope, intercept and bias, in response to high and low glucose levels, of a test strip lot may be impacted by varying the enzyme ink pad height printed onto the strips. More specifically, it is a discovery of the invention that the slope, intercept and bias of a test strip lot may be linearly increased by increasing the enzyme pad height, and decreased by decreasing the height, thus providing an improved method for the production of single calibration code strip lots with good yields.

In one embodiment the invention provides a method of manufacturing a test strip batch in which the enzyme pad height is selected so that the slope, intercept, and bias of the batch falls within a predetermined target for a predetermined calibration code. The method comprises, consists essentially of and consists of: (a) selecting a desired slope, intercept and bias for a batch of test strips; and (b) computing an emulsion thickness to be used in a screen for printing an enzyme ink based on the desired slope, intercept and bias and a previous batch slope, intercept and bias obtained from a previously made test strip batch so that a resulting slope, intercept and bias is substantially equal to the desired slope, intercept and bias.

For purposes of the invention, a “batch” of test strips is a set of strips made using one roll of substrate A roll of substrate is a continuous piece of substrate that may or may not be spliced with one or more other rolls of substrate to form a continuous web of substrate. The roll, typically, after printing is separated into cards and again into test strips

By “bias” is meant the difference between a measured glucose value and an accepted reference glucose value. Absolute bias is measured in units of mg/dL. Relative bias, or pbias, is expressed as a percent of the absolute bias value over the reference value.

By “batch slope” or “slope” is meant the slope value for the batch of test strips and by “batch intercept” or “intercept” is meant the intercept value for the batch.

For purposes of the invention, by “pad height” is meant the enzyme layer thickness on a test strip. The enzyme layer of a test strip typically is printed onto the strip using mesh printing screens. The screen is composed of mesh material suitable for use in the printing process to be used. Suitable screen materials include nylon, polyester and stainless steel. Preferably, the material is a polyester.

For purposes of printing the enzyme layer, one side of the mesh material is coated with an emulsion by any conveneinet method, which emulsion incorporates a pattern for the enzyme ink layer to be printed onto a substrate. The pattern can be formed in the emulsion by any convenient method including applying the emulsion to one side of the mesh material, curing portions of the emulsion, and removing the uncured portions of the emulsion so that the emulsion layer with the pattern remains on the mesh.

The emulsion may be applied to the mesh by any known method such as by using a coating trough and pressing the trough against the screen onto which liquid emulsion has been placed and drawing the trough along the screen. The emulsion thickness will depend on the pressure applied during this process along with the speed at which the trough is drawn over the mesh, the trough angle, the mesh type and count and the screen size. This process may be carried out multiple times to achieve the desired thickness. Alternatively, a stencil of the image to be printed may be made on an emulsion film and the film is then pressed onto the mesh. As yet another alternative, an emulsion film may be attached to the mesh by putting a coating of emulsion on the other side of the film to be attached to the mesh, after which the applied emulsion coating is cured.

The invention may find its greatest utility in the manufacture of electrochemical-based test strips for the determination of glucose levels in whole blood samples. More preferably, the invention is used in electrochemical test strips for measuring glucose, which electrodes have co-planar electrodes. Most preferably the inks of the invention are used in ULTRA™ type test strips as disclosed in U.S. Pat. Nos. 5,708,247, 5,95,836, 7,112,265, 6241,862, 6284,125, 7,462,265 and U.S. Patent Publication Nos. 20100112678 and 20100112612, incorporated herein in their entireties by reference.

For purposes of printing the enzyme layer onto the test strip substrate, the pattern is printed using any suitable method. In one such method a movable, generally flat screen is used. A suitable print station in such a process may include a screen, print rollers, flood blade, and squeegee. The screen is charged with enzyme ink, on the side of the screen opposite of the side with the emulsion and pattern to be printed, by moving the squeegee, flood blade and roller in a first direction corresponding to the web movement of the substrate. The screen is then moved in a second direction opposite of the first for a flood cycle during which ink is charged onto the screen. The ink is then transferred by the squeegee through the screen and onto the substrate. An embodiment of a suitable printing mechanism is described in U.S. Pat. No. 4,245,554 which is incorporated in its entirety by reference herein.

In the method of the invention, the emulsion thickness of the screen is selected to obtain the desired slope, intercept and bias for a batch of test strips. It is a discovery of the invention that, by increasing emulsion thickness on the mesh printing screen to obtain an increase in the resulting enzyme pad height printed onto a batch of test strips, there will be a corresponding linear increase in slope, intercept and bias of the test strip batch compared with a previously produced batch of test strips using the same enzyme ink. By increasing the emulsion thickness printed onto the substrate by about 1 μm, a change of about 0.45 μm in the printed pad height will result. Screens with different emulsion thicknesses may be commercially obtained from screen manufacturers.

The bias of the strips of the invention may be calibrated by any convenient method including without limitation, the following method. An amount, typically around 1500 strips, are selected at random from the batch. Blood from 12 different donors is spiked to each of six levels of glucose and eight strips are given blood from identical donors and levels so that a total of 12×6×8=576 tests are conducted for that batch. These are benchmarked against actual blood glucose concentration by measuring these using a standard laboratory analyzer such as Yellow Springs Instrument (“YSI”). A graph of measured glucose concentration is plotted against actual glucose concentration (or measured current versus YSI current), and a formula y=m×+c least squares fitted to the graph to give a value for batch slope m and batch intercept c for the remaining strips from the lot or batch. The difference in response to high and low blood glucose contents may be described by any method that measures bias change over an operational range. For example, the linearity may be described by the term a in the quadratic calibration ax̂2+mx+c, wherein m is the slope and c is the slope intercept.

After emulsion thickness is set, a verification run may be performed to verify that a linearity substantially equal to the desired slope, intercept and bias values is achieved. If the linearity is substantially equal to the target values, then the method will move forward to large-scale production batches. However, if the second linearity is not substantially equal to the target range, then the emulsion thickness used is adjusted and more strips prepared and tested to verify that the modified size provides the values that are desired. This process can be repeated as necessary.

It should be noted that other factors including, without limitation, the amount of mediator, the conductive ink lot, oxidized mediator lot, mixing time, mixing process, standing time, preconditioning of substrate, mesh type, mesh deformability, working electrode length, working electrode area, working electrode separation and snap distance, may affect one or both of the batch slope and intercept. These can be controlled so as to be sufficiently identical during each run such that a substantially constant slope and intercept are obtained batch-to-batch. Preferably, the working electrode area and the amount of reduced mediator are controlled, as described in United States Patent Publication No. 20090208743A1 incorporated herein in its entirety by reference, so as to achieve a substantially constant slope and intercept.

A test strip using the invention may be manufactured using any convenient, known method including, without limitation, web printing, screen printing and combinations thereof. For example, the strip may be manufactured by sequential, aligned formation of a patterned conductor layer, insulation layer, reagent layer, patterned adhesive layer, hydrophilic layer and a top film onto an electrically insulating substrate.

An exemplary web printing process is as follows. A substrate is used that may be nylon, polycarbonate, polyimide, polyvinyl chloride, polyethylene, polypropylene, glycolated polyester, polyester and combinations thereof. Preferably, the substrate is a polyester, more preferably Melinex ST328, which is manufactured by DuPont Teijin Films. Prior to entering one or more printing stations, the substrate may be preconditioned to reduce the amount of expansion and stretch that can occur in the strip manufacturing process. In the preconditioning step, the substrate may be heated to a temperature, which is not exceeded in the subsequent print steps. For example, the substrate may be heated to approximately 160° C. Generally, the heating takes place under tension of between about 150N and 180N more typically around 165N. Alternatively, preconditioning the substrate can be heated to a temperature sufficient to remove the irreversible stretch, again optionally while under tension as described above.

Preferably, the substrate is held under a tension of approximately 165N throughout the process in order to maintain registration of the layers to be printed. The substrate is also subjected to various temperatures of about 140° C. or less in order to dry the printed inks during each printing step. Optionally, prior to printing a cleaning system may be used which cleans the top, or print, side and the underside of the substrate using a vacuum and brush system.

One or more prints with carbon with metallic particles, silver/silver chloride ink or gold or palladium based inks or any combination thereof in one or more printing steps may be used to provide an electrode layer. In one embodiment, prior to the printing process and immediately after drying, the substrate is passed over a first chilled roller, to rapidly cool the substrate to a predetermined temperature, typically room temperature around 18-21° C. and typically 19.5° C.+/−0.5° C. After the printed carbon patterns are deposited in the printing process, the substrate may be passed over a second chilled roller.

Any ink suitable for use as an insulation ink and applicable in a print station in a web manufacturing process may be used including, without limitation, Ercon E6110-116 Jet Black Insulayer Ink, which may be purchased from Ercon, Inc. Immediately after drying, the substrate, including printed carbon and insulation patterns, is passed over third chilled roller as described above.

A first enzyme ink printing may then take place using an ink of the invention. After the first enzyme ink printing process and immediately after drying, the substrate, including printed carbon and insulation patterns, is passed over a fourth chilled roller. One or more of a topside, underside and side humidification may be provided. For example, an arrangement of pipes may provide a substantially constant stream of humidified air above, below and sideways onto the substrate and layers ensuring the water content of the ink is maintained at a constant level. The amount and arrangement of humidification, typically pipes carrying humidified air, will depend, amongst other things, upon the amount of humidification required, the water content of the ink, the humidity and temperature of the surrounding air, the temperature of the substrate as it approaches the enzyme print station, the temperature of the print roller, the size of the screen and the exposure of the screen to the surrounding, unhumidified air.

An exemplary test strip may include multiple layers disposed on a substrate. The seven layers disposed on the substrate may be a conductive layer, which can also be referred to as electrode layer, an insulation layer, one or more overlapping reagent layers, an adhesive layer, a hydrophilic layer, and a top layer.

The conductive layer may include a reference electrode, first and second working electrodes, first and second contact pads, a reference contact pad, first and second working electrode tracks, a reference electrode track, and a strip detection bar. The conductive layer may be formed from carbon ink. The first contact pad, second contact pad, and reference contact pad may be adapted to electrically connect to a test meter. The first working electrode track provides an electrically continuous pathway from first working electrode to first contact pad. Similarly, the second working electrode track provides an electrically continuous pathway from the second working electrode to the second contact pad. Similarly, the reference electrode track provides an electrically continuous pathway from the reference electrode to the reference contact pad. The strip detection bar is electrically connected to reference contact pad. A test meter can detect that the test strip has been properly inserted by measuring a continuity between the reference contact pad and the strip detection bar.

The enzyme ink layer may be disposed on a portion of the conductive layer, substrate, and insulation layer. In one embodiment, two successive enzyme ink layers may be screen-printed on the conductive layer, typically also overlapping slightly insulation layer. The adhesive layer may include first, second and third adhesive pads and may be deposited on the test strip fter the deposition of the reagent layer. The first and second adhesive pads can be aligned to be immediately adjacent to, touch, or partially overlap with the reagent layer. The adhesive layer may include a water based acrylic copolymer pressure sensitive adhesive which is commercially available from Tape Specialties LTD, which is located in Tring, Herts, United Kingdom. The adhesive layer is disposed on a portion of insulation layer, conductive layer, and substrate and binds the hydrophilic layer to the test strip.

The hydrophilic layer may include distal and proximal hydrophilic portions and may be a polyester having one hydrophilic surface such as an anti-fog coating, which is commercially available from 3M. The final layer to be added to the test strip is a top layer that may include a clear portion and opaque portion. The top layer is disposed on and adhered to the hydrophilic layer. The top layer may be a polyester that has an adhesive coating on one side

The invention will be further clarified by a consideration of the following, non-limiting examples.

### EXAMPLES

### Example 1

Commercially available polyester screens for printing of an enzyme layer onto a test strip substrate were obtained with emulsion thicknesses of 6, 13, and 17 μm, respectively. The web printing process described above was used to produce three test strips batches. Ferricyanide absorbance testing at 420 nm using an Ultrospec 2100 Pro UV/Vis spectrophotometer was used to inferred the enzyme pad height. The testing was carried out by placing a strip into a container hodling 1 cc of purified water, such as ANALAR™, and leaving the container with the strips and water in a space that was substantially without ambient light for approximately 10 minutes and then stirring for approximately 5 secs. using a fixed speed vortex mixer after which 1 ml from each sample container was measured in the spectrophotometer. The data was then used to calculate enzyme thickness using the following calculations:

Concentration of ferricyanide(M)=absorbance/extinction coefficient for potassium ferrocyanide(Lmol⁻¹cm⁻¹)

Ferri(mol/strip)=concentration of ferricyanide(m)/1000

Ferri(g/strip)=Ferri(mol/strip)*MW of potassium ferrocyanide(g/mol⁻¹)

Ferri volume(cm³/strip)=ferri(g/strip)/density of potassium ferrocyanide (g/cm³)

Enzyme volume(cm³/strip)=ferri volume(cm³/strip)*(100/volume percent potassium ferricyanide in dried enzyme ink

Enzyme height(cm)=enzyme volume(cm³/strip)/(length of enzyme print(cm)*width of enzyme print(cm))

Enyzme height(nm)=enzyme height(cm)/1000

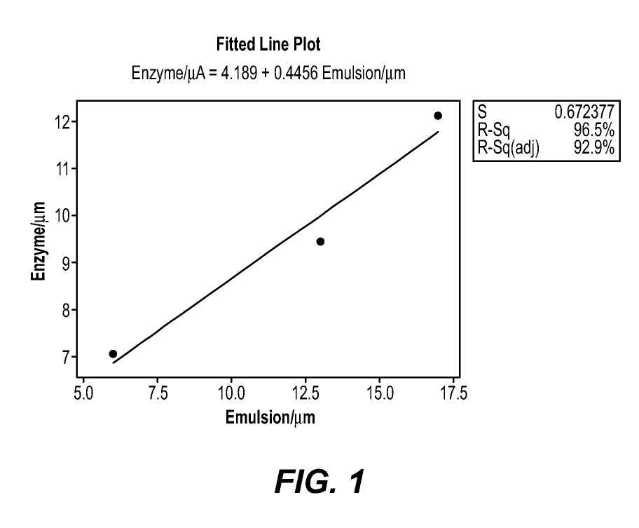

The enzyme pad height was then plotted against the emulsion thickness, as shown in FIG. 1. Using regression analysis commercially available linear regression analysis software it can be seen that an approximately 1 nm change in emulsion thickness results in an approximately 0.45 nm change in enzyme pad height

The strips were calibrated by randomly selecting 1500 strips. Blood from 12 different donors was spiked to each of 6 levels (50, 100, 150, 200, 300 and 500 mg) of glucose and 8 strips were given blood from identical donors and levels so that a total of 12×6×8 or 576 tests were conducted for each test batch. These were benchmarked against actual blood glucose concentration by measuring these using a standard laboratory analyzer, a Yellow Springs instrument 2300 (“YSI”). A graph of measured glucose concentration was plotted against actual glucose concentration (or measured current versus YSI current) and a formula y=m×+c least squares fitted to the graph to give a value for batch slope m and batch intercept c.

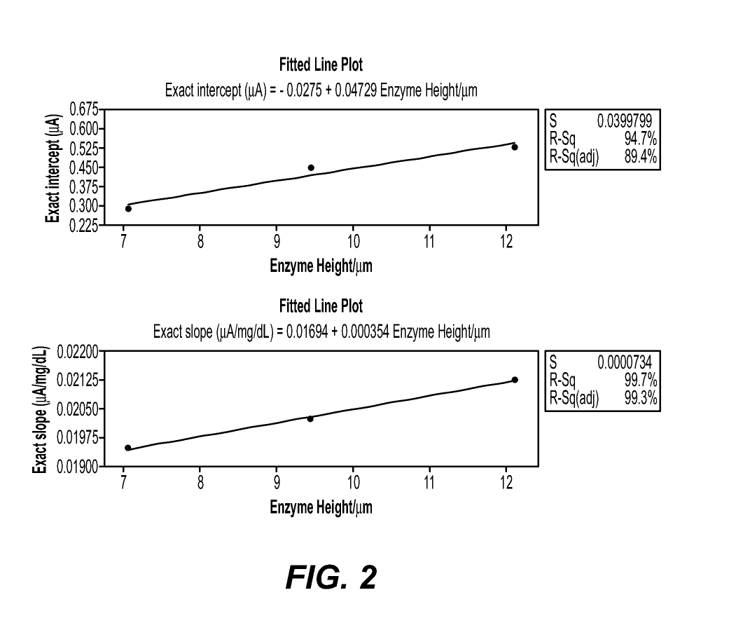

The slopes and intercepts are listed on the Table below and a graph of the plot of the intercepts and slopes are shown in FIG. 2.

TABLE 2

| | Emulsion | | | Exact |
|---|---|---|---|---|
| | Thickens | Calibration | Exact Slope | Intercept |
| Batch | (μm) | Code | (μA/mg/dL) | (μA) |
| 3051413 | 17 | 46 | 0.0213 | 0.530 |
| 3051414 | 6 | 22 | 0.0195 | 0.289 |
| 3051416 | 13 | 25 | 0.0202 | 0.452 |

The intercept and slope plot shows that, as enzyme pad height increase, slope and intercept increase linearly. The R-sq values indicate a very strong correlation between intercept and enzyme pad height and slope and enzyme pad height.

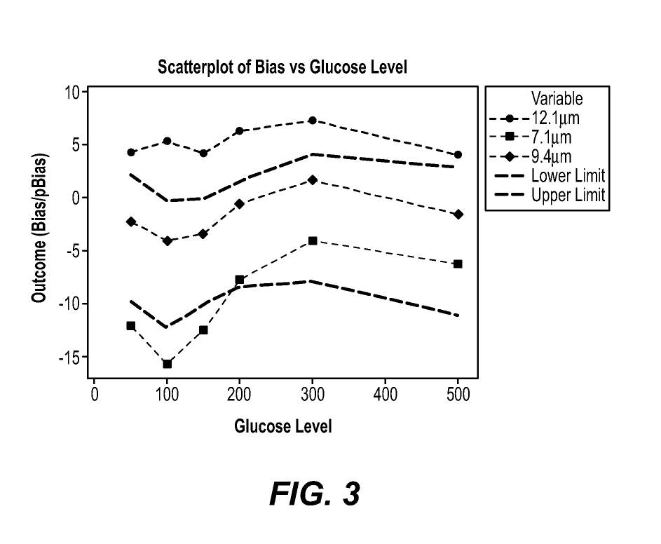

In addition, the bias and percent bias levels were calculated for each glucose level. A scatterplot of bias or percent bias versus glucose level is shown in FIG. 3. As can be seen, bias at each glucose level becomes more positive linearly as emulsion thickness, and enzyme print height, increases.

The results demonstrate that changing the enzyme pad height by altering the printing mesh and, thus, the emulsion thickness results in a significant change in strip performance. Thus, it is possible to use emulsion thickness to later the performance of a test strip batch into a single calibration code region.

What we claim is:

1. A method of manufacturing a test strip batch, comprising (a) selecting a desired slope, intercept and bias for a batch of test strips; and (b) computing an emulsion thickness to be used in a screen for printing an enzyme ink onto a test strip substrate based on the desired slope, intercept and bias and a previous batch slope, intercept and bias obtained from a previously made test strip batch so that a resulting slope, intercept and bias is substantially equal to the desired slope, intercept and bias.
